(19) United States
(12) Patent Application Publication
(10) Pub. No.: US 2026/0274869 A1
(43) Pub. Date: Sep. 17, 2026

(54) PREPARATION METHODS OF ETHER-BONDED PORPHYRIN DIMER ESTER AND SINOPORPHYRIN SODIUM, PHARMACEUTICAL COMPOSITIONS CONTAINING THEM AND THEIR USES

(71) Applicant: SHANGHAI GUANGSHENG BIOPHARMACEUTICAL CO., LTD, Shanghai (CN)

(72) Inventors: Shaoliang ZHANG, Shanghai (CN); Junhui ZHANG, Shanghai (CN); Yiwen LIU, Shanghai (CN)

(73) Assignee: SHANGHAI GUANGSHENG BIOPHARMACEUTICAL CO., LTD, Shanghai (CN)

(21) Appl. No.: 19/673,098

(22) Filed: May 11, 2026

Related U.S. Application Data
(63) Continuation of application No. PCT/CN2024/077339, filed on Feb. 18, 2024.

(51) Int. Cl. C07D 519/00

(52) U.S. Cl. CPC C07D 519/00

(57) ABSTRACT

The present disclosure relates to a method for preparing ether-bonded porphyrin dimer ester, which comprises the compound of protoporphyrin IX dimethyl ester in formula (III) is reacted at −10-10° C. in the presence of haloalkane solvent, hydrogen bromide gas, and oxygen, to obtain the compound of bis[1-[6,7-dipropionate methyl-1,3,5,8-tetramethyl-2-vinyl-4-porphyrin]ethyl] ether in formula (II). Furthermore, this disclosure also relates to a method for preparing sinoporphyrin sodium. These preparation methods are characterized by mild reaction conditions, simplicity and easy operation, controllable quality, and suitability for industrial production, the products prepared by these methods exhibit high purity, good yield, longer shelf life, and good storage stability.

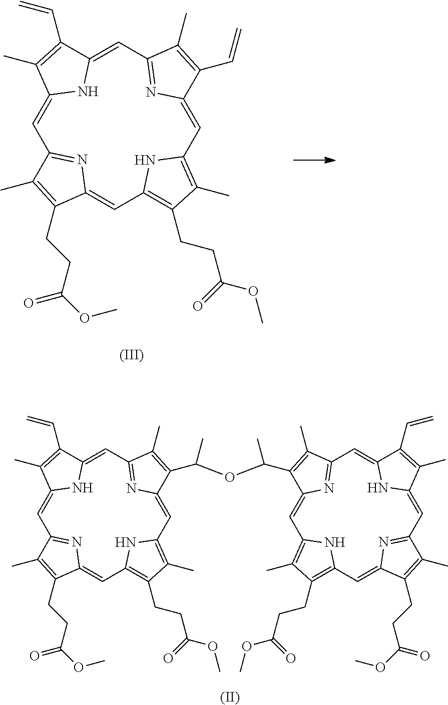

### CROSS-REFERENCE TO RELATED APPLICATION

This application is a continuation of International Patent Application No. PCT/CN2024/077339 filed on Feb. 18, 2024, the disclosure of which is hereby incorporated by reference in its entirety.

### TECHNICAL FIELD

The present application belongs to the field of medicinal chemistry, and specifically relates to a method for preparing ether-bonded porphyrin dimer ester, a method for preparing sinoporphyrin sodium, and the ether-bonded porphyrin dimer ester and sinoporphyrin sodium prepared by said methods, pharmaceutical compositions containing them, and their uses as photosensitizers in photodynamic therapy.

### BACKGROUND

Photodynamic therapy (PDT) is a novel therapy that uses photosensitizers, light, and oxygen molecules to generate photodynamic reactions, thereby selectively curing diseases such as malignant tumors, vascular lesions, and microbial infections, wherein the photosensitizers are the core of photodynamic therapy. Currently, the photosensitizers used in clinical practice mainly include Photofrin from the United States, Photogem from Russia, Photosan from Germany, and Haematodrex from Belgium, etc. However, these photosensitizers still have many disadvantages. For example, they are mostly mixed preparations composed of porphyrin derivatives, the active ingredients are unclear, there are no controllable quality standards, and because they remain in the skin for several weeks, they are prone to causing skin phototoxicity such as rashes and blisters. Patients need to avoid direct sunlight for one month or even longer after administration (Fang Qicheng. Photodynamic therapy for cancer and new anticancer photosensitizer sinoporphyrin sodium [J], Chinese Journal of New Drugs, 2014, 23 (13): 1540-1545).

Sinoporphyrin sodium (DVDMS), as a new generation of photosensitizer independently developed in China, is favored by people for features such as its well-defined active ingredients, good water solubility, high content, low effective dose, short light avoidance time after treatment, good safety, and unique fluorescence properties, etc. Given its broad application prospects in anti-tumor, antibacterial, psoriasis treatment, and the development of therapeutic and diagnostic reagents, etc. the large-scale production of sinoporphyrin sodium with high purity and good yield is crucial to meet the growing market demand and increasingly stringent application requirements. However, existing methods for preparing sinoporphyrin sodium suffer from disadvantages such as low yield, complex processes, and low product purity, etc. Thus, there remains a need for efficient, large-scale processes to prepare sinoporphyrin sodium in high purity and high yield.

### SUMMARY

To address the aforementioned problems, the present disclosure aims to provide a method for the large-scale preparation of bis[1-[6,7-dipropionate methyl-1,3,5,8-tetramethyl-2-vinyl-4-porphyrin]ethyl] ether with good yield and high purity, a method for preparing sinoporphyrin sodium, and the ether-bonded porphyrin dimer ester and sinoporphyrin sodium prepared by said methods, pharmaceutical compositions containing them, and their uses as photosensitizers in photodynamic therapy.

A first aspect of the present disclosure provides a method for preparing the compound of bis[1-[6,7-dipropionate methyl-1,3,5,8-tetramethyl-2-vinyl-4-porphyrin]ethyl] ether in formula (II) (hereinafter also referred to as “ether-bonded porphyrin dimer ester”), the method comprising:

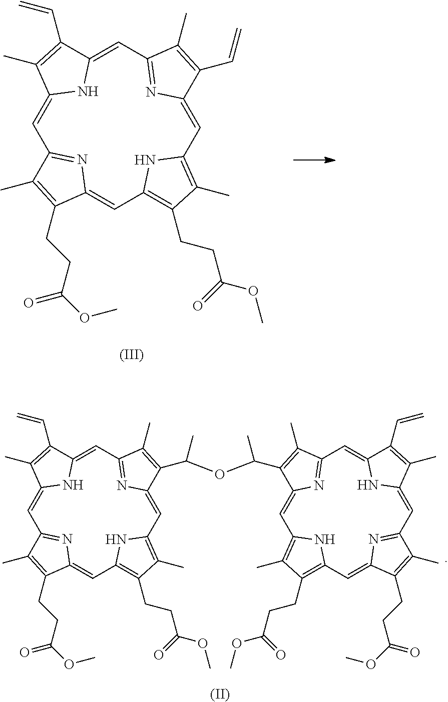

The compound of protoporphyrin IX dimethyl ester in formula (III) is reacted at −10-10° C. in the presence of haloalkane solvent, hydrogen bromide gas, and oxygen to obtain the compound of bis[1-[6,7-dipropionate methyl-1,3,5,8-tetramethyl-2-vinyl-4-porphyrin]ethyl] ether in formula (II).

Unexpectedly, the inventors discovered that the method for preparing the compound of ether-bonded porphyrin dimer ester in formula (II) of the present disclosure directly uses protoporphyrin IX dimethyl ester as a raw material and adopts a simple “one-step method”, which greatly simplifies the process (e.g., forming protoporphyrin IX dimethyl ester derivatives, separating various protoporphyrin IX dimethyl ester derivatives, etc.), reduces production costs, reduces product loss caused by cumbersome intermediate processing, and can be directly used in solid or solution form to synthesize sinoporphyrin sodium in a quality suitable for preparing sinoporphyrin sodium (e.g., with good yield and high purity), thus making it more suitable for industrial production.

A second aspect of the present disclosure provides a method for preparing the compound of sinoporphyrin sodium in formula (I) (i.e., bis[1-[6,7-dipropionate sodium salt-1,3,5,8-tetramethyl-2-vinyl-4-porphyrin]ethyl] ether), the method comprising the following steps:

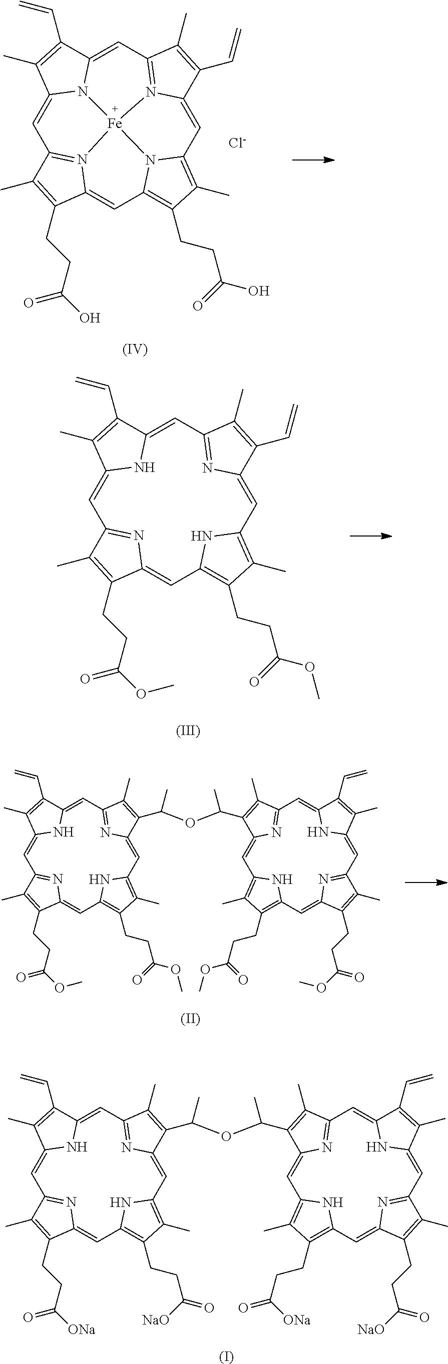

(S1) The compound of hemin chloride in formula (IV) is reacted with methanol and reduced iron powder in the presence of haloalkane solvent and hydrogen chloride gas to obtain the compound of protoporphyrin IX dimethyl ester in formula (III);
(S2) The compound of bis[1-[6,7-dipropionate methyl-1,3,5,8-tetramethyl-2-vinyl-4-porphyrin]ethyl] ether in formula (II) is obtained from the compound in formula (III) according to the method of the first aspect of the present disclosure;
(S3) The compound of bis[1-[6,7-dipropionate methyl-1,3,5,8-tetramethyl-2-vinyl-4-porphyrin]ethyl] ether in formula (II) is reacted with sodium hydroxide C₁-C₃ fatty alcohol solution in the presence of haloalkane solvent to obtain the compound of sinoporphyrin sodium in formula (I).

According to the method for preparing the compound of sinoporphyrin sodium in formula (I) of the present disclosure, the method may further comprise a step (S4) of purification by preparative high performance liquid chromatography (HPLC).

According to the method for preparing the compounds in formula (I) and formula (II) of the present disclosure, the haloalkane solvent is selected from C₁-C₃ haloalkane, preferably, selected from at least one of dichloromethane, dichloroethane, trichloromethane, carbon tetrachloride, and dibromomethane, more preferably, dichloromethane.

The advantages of the above-described method for preparing the compound of sinoporphyrin sodium in formula (I) are that the raw materials are inexpensive and readily available, the method for synthesizing the necessary intermediates is simple and easy to operate, requirements for the equipment at each stage are low, the reaction conditions are mild, and the quality is controllable, which is conducive to reducing production costs and industrializing the production of sinoporphyrin sodium, and the sinoporphyrin sodium prepared by the said method has better quality, such as high purity, good yield, longer shelf life, and good storage stability, for example, a shelf life of more than 24 months, and its purity does not decrease significantly with the extension of storage time.

A third aspect of the present disclosure provides the compound of protoporphyrin IX dimethyl ester in formula (III), the compound of bis[1-[6,7-dipropionate methyl-1,3,5,8-tetramethyl-2-vinyl-4-porphyrin]ethyl] ether in formula (II), and the compound of sinoporphyrin sodium in formula (I), prepared according to the method of the second aspect of the present disclosure.

A fourth aspect of the present disclosure provides a pharmaceutical composition for photodynamic therapy, which comprises sinoporphyrin sodium prepared according to the method of the second aspect of the disclosure and one or more pharmaceutically acceptable excipients.

A fifth aspect of the present disclosure provides the uses of sinoporphyrin sodium prepared according to the method of the second aspect of the disclosure in the preparation of a medicament used as a photosensitizer in photodynamic therapy.

### DETAILED DESCRIPTION

Preferred embodiments of the present disclosure will be described in detail below. It should be noted that this application is not limited to these embodiments. These embodiments are merely exemplary, and those skilled in the art could make various modifications, additions, and substitutions to the present disclosure without departing from the scope and spirit of the present disclosure.

The “range” disclosed in this application is defined by a lower limit and an upper limit, a given range is defined by selecting a lower limit and an upper limit, which define the boundaries of a particular range. Ranges defined in this way may comprise or exclude endpoints and may be arbitrarily combined, that is, any lower limit may be combined with any upper limit to form a range. For example, if ranges of 60-120 and 80-110 are listed for a specific parameter, it is expected that ranges of 60-110 and 80-120 are also comprised. Furthermore, if minimum range values of 1 and 2 are listed, and if maximum range values of 3, 4, and 5 are listed, then the following ranges are all expected: 1-3, 1-4, 1-5, 2-3, 2-4, and 2-5. In this application, unless otherwise stated, the numerical range “a-b” represents a shortened representation of any combination of real numbers between a and b, where a and b are real numbers. For example, the numerical range “0-5” indicates that all real numbers between “0-5” have been listed herein, “0-5” is simply a shortened representation of these numerical combinations. Furthermore, when a parameter is stated as an integer≥2, it is equivalent to disclosing that the parameter is, for example, an integer such as 2, 3, 4, 5, 6, 7, 8, 9, 10, 11, 12, etc.

Unless otherwise specified, all embodiments and optional embodiments of this application may be combined to form new technical solutions.

Unless otherwise specified, all technical features and optional technical features of this application may be combined to form new technical solutions.

Unless otherwise specified, all steps of the present disclosure may be performed sequentially or randomly, preferably sequentially. For example, the method comprises steps (a) and (b), indicating that the method may comprise steps (a) and (b) performed sequentially, or it may also comprise steps (b) and (a) performed sequentially. For example, the method may also comprise step (c), indicating that step (c) may be added to the method in any order. For example, the method may comprise steps (a), (b), and (c), or it may comprise steps (a), (c), and (b), or it may also comprise steps (c), (a), and (b), etc.

Unless otherwise specified, the terms “comprise” and “include” as mentioned in this application can be open-ended or closed-ended. For example, “comprise” and “include” may mean that other members, elements, or method steps not listed may also be comprised or included, or may only comprise or include other listed members, elements, or method steps.

The “more than” and “less than” used in this application comprise the number itself. For example, “more than one” means one or more, and “one or more of A and B” means “A”, “B”, or “A and B”.

Unless otherwise specified, the term “or” is inclusive in this application. For example, the phrase “A or B” means “A, B, or both A and B”. More specifically, the condition “A or B” is satisfied by any of the following conditions: A is true (or exists) and B is false (or does not exist); A is false (or does not exist) and B is true (or exists); or both A and B are true (or exist).

In this disclosure, unless otherwise stated, all operations are performed under normal temperature and pressure conditions.

As used herein, “C₁-C₃ fatty alcohol” refers to a straight-chain or branched saturated aliphatic alcohol containing 1 to 3 carbon atoms, preferably a straight-chain or branched saturated aliphatic monohydric alcohol containing 1 to 3 carbon atoms, examples of which comprise, but are not limited to, methanol, ethanol, propanol or isopropanol.

As used herein, “C₁-C₃ haloalkane” refers to a straight-chain saturated C₁-C₃ alkyl group that is monosubstituted or polysubstituted with halogen atoms (e.g., chlorine, bromine), preferably polysubstituted, and examples of which comprise, but are not limited to, dichloromethane, dibromomethane, trichloromethane, tetrachloromethane or dichloroethane.

Unless otherwise specified, in this application, the expression “compound in formula (I)” may be used interchangeably with “bis[1-[6,7-dipropionate sodium salt-1,3,5,8-tetramethyl-2-vinyl-4-porphyrin]ethyl] ether” and “sinoporphyrin sodium”; the expression “compound in formula (II)” may be used interchangeably with “bis[1-[6,7-dipropionate methyl-1,3,5,8-tetramethyl-2-vinyl-4-porphyrin]ethyl] ether” and “ether-bonded porphyrin dimer ester”.

As used herein, the term “pharmaceuticalally acceptable excipient” refers to a substance that can be used to prepare a pharmaceutical composition and is generally safe, non-toxic, and free from undesirable properties in a biological or other sense, including excipients that are acceptable for veterinary and human pharmaceutical use.

Unless otherwise specified, all contents and percentages in the context of this application are based on weight.

Unless otherwise specified, all the water used in this application is purified water, which complies with the provisions of the Chinese Pharmacopoeia (2020 edition), Part II, to minimize the types and contents of impurities in the final product.

A first aspect of the present disclosure provides a method for preparing the compound of bis[1-[6,7-dipropionate methyl-1,3,5,8-tetramethyl-2-vinyl-4-porphyrin]ethyl] ether in formula (II), the method comprising:

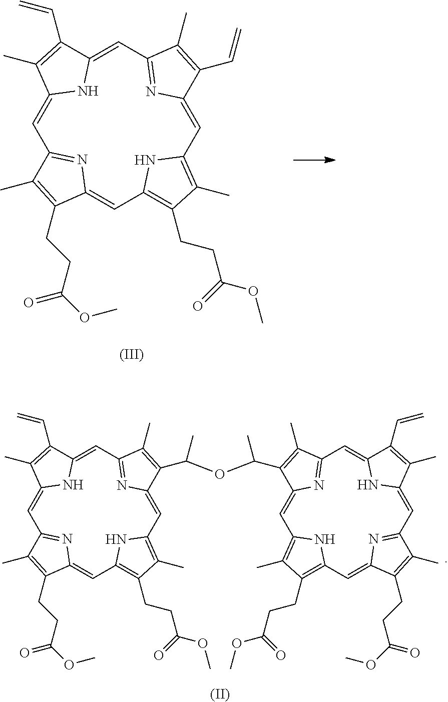

The compound of protoporphyrin IX dimethyl ester in formula (III) is reacted at −10-10° C. in the presence of haloalkane solvent, hydrogen bromide gas, and oxygen to obtain the compound of bis[1-[6,7-dipropionate methyl-1,3,5,8-tetramethyl-2-vinyl-4-porphyrin]ethyl] ether in formula (II).

In a preferred embodiment, the haloalkane solvent is selected from C₁-C₃ haloalkane, preferably, selected from at least one of dichloromethane, dichloroethane, trichloromethane, carbon tetrachloride, and dibromomethane, more preferably, dichloromethane.

In a preferred embodiment, the reaction is carried out at −10-10° C., preferably, −5-5° C., for 15 to 22 hours, preferably, 16 to 20 hours.

In a preferred embodiment, the weight ratio of the compound of protoporphyrin IX dimethyl ester in formula (III) to hydrogen bromide gas is 1:(0.7-1.4), preferably, 1:(0.75-1.3), more preferably, 1:(0.8-1.2).

In a preferred embodiment, the molar content of oxygen in the hydrogen bromide gas in the reaction system is 4%-6%, preferably, 4.5%-5.5% (based on the total amount of hydrogen bromide gas and oxygen gas). The oxygen may originate from oxygen already present in the reaction system, oxygen additionally introduced into the reaction system, etc., preferably, oxygen additionally introduced into the reaction system.

According to the method for preparing the compound in formula (II) of the present disclosure, the reaction is terminated by adding water at 20-30° C. Preferably, the amount of water added is 9-11 times that of protoporphyrin IX dimethyl ester, more preferably, 9.5-10.5 times.

According to the method for preparing the compound in formula (II), the amount of haloalkane solvent used is 10-18 times the total weight of protoporphyrin IX dimethyl ester and hydrogen bromide gas, preferably, 12-16 times, and more preferably, 13-15 times.

Preferably, in one specific embodiment, the amount of dichloromethane used is 10-18 times the total weight of protoporphyrin IX dimethyl ester and hydrogen bromide gas, more preferably, 12-16 times, and more preferably, 13-15 times.

According to the above method for preparing the compound in formula (II), the reaction apparatus used for the reaction could be a reaction apparatus conventionally used in the art, for example, a reaction vessel equipped with a stirrer and a temperature control unit.

According to the above method for preparing the compound in formula (II), the method may further comprise a post-treatment step of the reaction mixture, which may be selected from extraction, filtration, vacuum concentration, purification, and drying, etc. In a preferred embodiment, the method may further comprise a step of adjusting the pH of the system mixture before performing the post-treatment. In a more preferred embodiment, before performing the post-treatment, the pH of the system is adjusted using a 7% sodium bicarbonate solution at 20-30° C., preferably, to a pH of 5-9.

According to the above method for preparing the compound in formula (II), extraction is carried out at 20-30° C., if applicable. To further improve the purity of the target product, the mixture obtained from the reaction may be extracted one or more times as needed. In the case of multiple (preferably two) extractions, the extraction methods may be the same or different. In this disclosure, there are no particular limitations on the extraction apparatus used, as long as it can achieve the extraction purpose, for example, an extraction tower, a static mixer, a reaction apparatus with stirring, etc., preferably, a reaction vessel with a stirrer.

In a preferred embodiment, the method may further comprise an extraction step of the mixture obtained from the reaction, wherein water and dichloromethane are used for extraction, and the weight ratio of water to dichloromethane is 1:(1-1.5), preferably, 1:(1.1-1.3). By extracting and combining the organic phases, the loss of the compound of ether-bonded porphyrin dimer ester in formula (II) separated from the mixture can be reduced.

In a preferred embodiment, the method may further comprise a step of filtering the mixture obtained from the reaction. In a more preferred embodiment, the method may further comprise a step of extracting the mixture obtained from the reaction and then filtering the extracted organic phase.

According to the above method for preparing the compound in formula (II), if applicable, the mixture obtained from the reaction may be filtered more than once as needed, preferably, 1-3 times, more preferably, once. In the case of multiple filtrations, the filtration methods may be the same or different. The filtration methods comprise positive pressure filtration, vacuum filtration, centrifugal filtration, etc. More preferably, the filtration is performed using a positive pressure plate filter, preferably, a stainless steel plate filter. The membrane pore size of the filter is 5 μm to 20 μm, preferably, 8 μm to 15 μm.

In a preferred embodiment, the method may further comprise a step of concentrating the mixture obtained from the reaction under reduced pressure. In a more preferred embodiment, the method may further comprise a step of filtering the mixture obtained from the reaction and then concentrating it under reduced pressure, wherein the filtered filtrate may be concentrated under reduced pressure at a temperature not exceeding 25° C. to the final concentration volume to obtain a concentrated solution containing the compound of protoporphyrin IX dimethyl ester in formula (III).

Unless otherwise stated, the expression “final concentration volume” refers to the volume, measured in liters, at which the liquid to be concentrated is concentrated to 2 to 4 times the total mass (in kilograms) of the main reactants used in the reaction (e.g., excluding solvents used for washing the reaction apparatus or for post-treatment). For example, if 1 kg of the main reactants is added, the final concentration volume is 2 to 4 liters.

In this application, there are no particular limitations on the apparatus for vacuum concentration, as long as it can achieve the purpose of vacuum concentration. Preferably, the apparatus for vacuum concentration can be selected from falling film evaporators, natural circulation evaporators, vacuum distillation columns, rotary evaporators, reaction vessels for concentration purposes, etc., preferably, rotary evaporators or reaction vessels for concentration purposes, wherein the reaction vessel for concentration purposes is equipped with a stirrer, a temperature control unit, a pressure reducing device, and a secondary condenser, and the internal pressure of the reaction vessel is-0.09 to 0 MPa.

In a preferred embodiment, the method further comprises a step of purifying the mixture obtained from the reaction by chromatographic separation, wherein the purification is performed by preparative medium-pressure liquid chromatography (MPLC), wherein 10% acetone solution and dichloromethane are used as eluents. Preferably, the mixture obtained from the reaction is purified by preparative medium-pressure liquid chromatography (MPLC) more than once, preferably, 1-3 times, more preferably, 2 times.

In a more preferred embodiment, the method may further comprise a step of concentrating the reaction mixture under reduced pressure, and then purifying the concentrated solution twice using preparative medium-pressure liquid chromatography (MPLC). Preferably, after the first purification by preparative medium-pressure liquid chromatography (MPLC), a product solution containing the compound of ether-bonded porphyrin dimer ester in formula (II), a crossed solution containing the compound of ether-bonded porphyrin dimer ester in formula (II), and a product-free solution containing porphyrin derivative impurities are obtained. Optionally, the crossed solution containing the compound of ether-bonded porphyrin dimer ester in formula (II) may be washed with dichloromethane and concentrated under reduced pressure.

In another preferred embodiment, when the mixture obtained from the reaction is purified once by preparative medium-pressure liquid chromatography, the eluent used is a 10% acetone solution and dichloromethane, wherein the volume ratio of the 10% acetone solution to dichloromethane is 1:(4-9).

In a preferred embodiment, the product solution containing the compound of ether-bonded porphyrin dimer ester in formula (II) obtained by preparative medium-pressure liquid chromatography (MPLC) for the first purification is then purified a second time by 10% acetone solution and dichloromethane as the eluent, wherein the ratio of the amount (by volume) of 10% acetone solution to dichloromethane is 1:(6-14), preferably, 1:(7-12), and more preferably, 1:(8-10).

According to the above method for preparing the compound in formula (II), the compound of ether-bonded porphyrin dimer ester in formula (II) is separated from the mixture obtained by reaction by preparative medium-pressure liquid chromatography (MPLC) using a specific volume ratio of 10% acetone solution and dichloromethane set in a series manner as eluents. At the same time, some impurities in the target product are removed, and the relative purity of the target product in the mixture is increased from about 15% to more than 90%, which greatly improves the yield.

According to the above method for preparing the compound in formula (II), the method may further comprise washing and vacuum concentration steps after chromatographic separation.

In one specific implementation, the product solution containing the compound of ether-bonded porphyrin dimer ester in formula (II) obtained by preparative medium-pressure liquid chromatography (MPLC) is washed with dichloromethane at a temperature not exceeding 25° C. and concentrated under reduced pressure to 1/10 of the original volume to obtain a dichloromethane solution of the compound of ether-bonded porphyrin dimer ester in formula (II).

According to the above method for preparing the compound in formula (II), the method may optionally comprise a drying step, such as freeze drying, infrared drying, vacuum drying, etc. The apparatus for drying may be selected from a tray oven, a vacuum drying oven, and a freeze dryer, preferably, a vacuum drying oven.

Preferably, the dichloromethane solution of the compound of ether-bonded porphyrin dimer ester in formula (II) obtained after a secondary purification by preparative medium-pressure liquid chromatography and vacuum concentration can be directly used for the next step of synthesizing sinoporphyrin sodium without drying, which simplifies intermediate processing operations such as dissolution and filtration, etc., thus making it very suitable for the industrial production of sinoporphyrin sodium.

Optionally, the method further comprises a recovery step. In one specific embodiment, the gas stream distilled during the vacuum concentration process is condensed and recovered using a condenser, preferably, a condenser comprising a pre-condenser and a post-condenser, or by means of a vacuum distillation column.

A second aspect of the present disclosure provides a method for preparing the compound of sinoporphyrin sodium in formula (I) (i.e., bis[1-[6,7-dipropionate sodium salt-1,3,5,8-tetramethyl-2-vinyl-4-porphyrin]ethyl] ether), the method comprising the following steps:

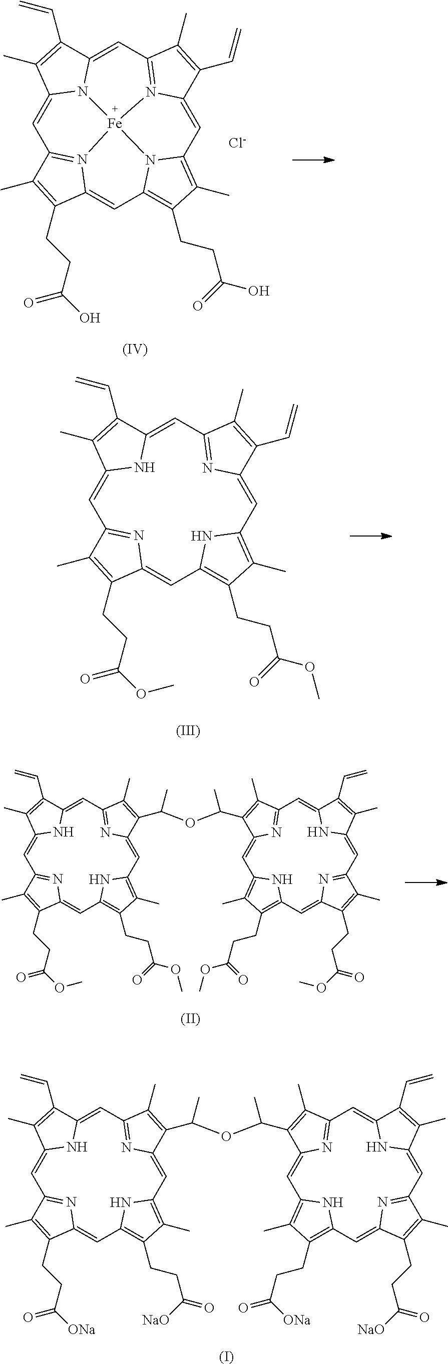

(S1) The compound of hemin chloride in formula (IV) is reacted with methanol and reduced iron powder in the presence of haloalkane solvent and hydrogen chloride gas to obtain the compound of protoporphyrin IX dimethyl ester in formula (III);
(S2) According to the method of the first aspect of the present disclosure, the compound of bis[1-[6,7-dipropionate methyl-1,3,5,8-tetramethyl-2-vinyl-4-porphyrin]ethyl] ether in formula (II) is obtained from the compound in formula (III);
(S3) The compound of bis[1-[6,7-dipropionate methyl-1,3,5,8-tetramethyl-2-vinyl-4-porphyrin]ethyl] ether in formula (II) is mixed and reacted with sodium hydroxide C₁-C₃ fatty alcohol solution in the presence of haloalkane solvent to obtain the compound of sinoporphyrin sodium in formula (I).

According to the method for preparing the compound of sinoporphyrin sodium in formula (I) of the present disclosure, the haloalkane solvent is selected from C₁-C₃ haloalkane, preferably, selected from at least one of dichloromethane, dichloroethane, trichloromethane, carbon tetrachloride, and dibromomethane, more preferably, dichloromethane.

According to the method for preparing the compound of sinoporphyrin sodium in formula (I) of the present disclosure, the method may further comprise a step (S4) of purifying by preparative high performance liquid chromatography (HPLC).

Unexpectedly, the inventors discovered that the method for preparing sinoporphyrin sodium of the present disclosure has many beneficial effects. For example, the reaction raw materials are inexpensive and readily available, the method for synthesizing the necessary intermediates with a quality suitable for the synthesis of sinoporphyrin sodium is simple and easy to operate, requirements for the equipment at each stage are low, the reaction conditions are mild, which helps to reduce production costs and is very suitable for the industrial production of sinoporphyrin sodium. Furthermore, the sinoporphyrin sodium prepared by the method has better quality, such as high purity, good yield, longer shelf life and good storage stability, for example, a shelf life of more than 24 months, and its purity does not decrease significantly with the extension of storage time.

The following will provide further detailed explanations of each synthesis stage.

### Step (S1)

According to the above method for preparing the compound of sinoporphyrin sodium in formula (I), in step (S1), the compound of hemin chloride in formula (IV) is reacted with methanol and reduced iron powder in the presence of haloalkane solvent and hydrogen chloride gas to obtain the compound of protoporphyrin IX dimethyl ester in formula (III).

In a preferred embodiment, in step (S1), the reaction is carried out at 18-30° C., preferably, 20-30° C., for 1-7 hours, more preferably, 2-6.5 hours, and more preferably, 3-6 hours.

In a preferred embodiment, in step (S1), the haloalkane solvent is selected from C₁-C₃ haloalkane, preferably, selected from at least one of dichloromethane, dichloroethane, trichloromethane, carbon tetrachloride, and dibromomethane, more preferably. dichloromethane.

In a preferred embodiment, in step (S1), the weight ratio of the compound of hemin chloride in formula (IV), reduced iron powder, hydrogen chloride gas and methanol is 1:(0.045-0.065):(1.08-1.35):(7.7-8.0), preferably, 1:(0.048-0.06):(1.1-1.32):(7.8-8.0), more preferably, 1:(0.05-0.06):(1.1-1.3):(7.8-7.9).

In some embodiments, in step (S1), the reaction is carried out at a reaction pressure not exceeding 0.06-0.09 MPa, preferably, not exceeding 0.03-0.05 MPa, and more preferably, 0.01 MPa. According to the method for preparing the compound in formula (I) of the present disclosure, excessive pressure may rupture the rupture disc of the reaction apparatus, potentially leading to reactant leakage and thus adversely affecting the environment, while excessive temperature may lead to increased impurities, reduced yield, and impact on economic efficiency.

In some embodiments of the present disclosure, when the reaction in step (S1) is completed, the ratio of the peak areas of hemin chloride to protoporphyrin IX dimethyl ester as determined by high performance liquid chromatography (HPLC) is ≤3%, preferably, ≤2%, and more preferably, ≤1%.

In a preferred embodiment, in step (S1), the reduced iron powder is added in batches, preferably, in three batches.

In a preferred embodiment, in step (S1), the amount of solvent used for the reaction is 1-2 times, preferably, 1-1.8 times, the total weight of hemin chloride, reduced iron powder, hydrogen chloride gas and methanol.

In this application, in step (S1), the reaction is carried out under an inert gas atmosphere. Preferably, the inert gas may be selected from at least one of nitrogen and argon, preferably, nitrogen.

According to the method for preparing the compound in formula (I) of the present disclosure, the reaction apparatus used for the reaction is as defined in the first aspect of the present disclosure above.

According to the method for preparing the compound in formula (I) of the present disclosure, step (S1) may further comprise a post-treatment step of the reaction mixture, which may be selected from extraction, filtration, washing, vacuum concentration, and drying. In a preferred embodiment, step (S1) may further comprise a step of adjusting the pH of the system mixture before performing the post-treatment. In a more preferred embodiment, triethylamine is used to adjust the pH of the system at 10-20° C.

In a preferred embodiment, step (S1) may further comprise an extraction step of the mixture obtained from the reaction, wherein water is used for extraction to obtain an organic phase containing protoporphyrin IX dimethyl ester and an aqueous phase containing iron salt, and the organic phase is separated. Preferably, the organic phase separated after extraction may be subjected to a second extraction in the same manner.

Unless otherwise stated, the general description of the extraction involved in step (S1) is as defined in the first aspect of the disclosure above.

In a preferred embodiment, when the mixture obtained from the reaction is subjected to a second extraction, the pH of the system is adjusted to 1-3 using triethylamine before the first extraction, and the pH of the system is adjusted to 7-8 using triethylamine before the second extraction.

In a preferred embodiment, step (S1) may further comprise a step of concentrating the resulting mixture under reduced pressure. In a more preferred embodiment, step (S1) may further comprise a step of filtering and then concentrating the resulting mixture under reduced pressure. Unless otherwise stated, the general description of the filtration step involved in step (S1) is as defined in the first aspect of the disclosure above.

In a preferred embodiment, step (S1) may further comprise a washing step after concentrating the reaction mixture under reduced pressure, wherein the washing is performed using dichloromethane and methanol, followed by drying at a temperature below 35° C., preferably, 20-30° C. Preferably, the drying apparatus used may be a tray oven, a vacuum drying oven, or a freeze dryer, preferably, a vacuum drying oven.

### Step (S2)

According to the above method for preparing the compound of sinoporphyrin sodium in formula (I), in step (S2), the compound of protoporphyrin IX dimethyl ester in formula (III) is reacted at −10-10° C. in the presence of haloalkane solvent, hydrogen bromide gas and oxygen to obtain the compound of ether-bonded porphyrin dimer ester in formula (II).

The various features, parameters, conditions, other elements and combinations thereof involved in step (S2) are as defined by the method for preparing the compound in formula (II) according to the first aspect of the present invention.

### Step (S3)

According to the above method for preparing the compound of sinoporphyrin sodium in formula (I), in step (S3), the compound of ether-bonded porphyrin dimer ester in formula (II) (i.e., bis[1-[6,7-dipropionate methyl-1,3,5,8-tetramethyl-2-vinyl-4-porphyrin]ethyl] ether) is mixed and reacted with a sodium hydroxide C₁-C₃ fatty alcohol solution in the presence of haloalkane solvent to obtain the compound of sinoporphyrin sodium in formula (I).

In a preferred embodiment, in step (S3), the reaction is carried out at 15-30° C., preferably, 15-25° C., for 6-18 hours, preferably, 8-16 hours.

In a preferred embodiment, in step (S3), the C₁-C₃ fatty alcohol is selected from methanol, ethanol, propanol or mixtures thereof, preferably, methanol.

According to the method for preparing sinoporphyrin sodium of the present disclosure, in step (S3), the weight ratio of the compound of the ether-bonded porphyrin dimer ester in formula (II) to the sodium hydroxide C₁-C₃ fatty alcohol solution is 1:(10-15), preferably, 1:(11-14), wherein the weight ratio of sodium hydroxide to C₁-C₃ fatty alcohol is 1:(13-22), preferably, 1:(15-20), more preferably, 1:(16-18).

In one specific embodiment, in step (S3), the weight ratio of the compound of ether-bonded porphyrin dimer ester in formula (II) to the sodium hydroxide methanol solution is 1:(10-15), preferably, 1:(11-14), wherein the weight ratio of sodium hydroxide to methanol is 1:(13-22), preferably, 1:(15-20), and more preferably, 1:(16-18).

According to the method for preparing sinoporphyrin sodium of the present disclosure, in step (S3), the amount of haloalkane solvent used for dissolution is 1-30 times, preferably, 2-29 times, and more preferably, 3-28 times, the weight of the ether-bonded porphyrin dimer ester.

In one specific embodiment, in step (S3), the amount of dichloromethane used for dissolution is 1-30 times, preferably, 2-29 times, and more preferably, 3-28 times, the weight of the ether-bonded porphyrin dimer ester.

In a preferred embodiment, step (S3) may further comprise a step of filtering the mixture obtained from the reaction. The filtration is performed via vacuum filtration by a filter. The filter is a tower filter with a pore size of 5-20 μm, preferably, 8-15 μm. Specifically, during the filtration operation, the mixture is washed with anhydrous ethanol to obtain an ethanol solution containing the compound of sinoporphyrin sodium in formula (I). Preferably, the ethanol solution containing sinoporphyrin sodium is dried under reduced pressure in a vacuum drying oven at a temperature not exceeding 45° C., preferably, 20-40° C. Optionally, the ethanol solution containing sinoporphyrin sodium may also be concentrated under reduced pressure before drying.

### Step (S4)

In a preferred embodiment, the method further comprises a step of purification by preparative high performance liquid chromatography (HPLC). In this step, the the compound of sinoporphyrin sodium in formula (I) obtained in step (S3) is dissolved in a mixture of methanol and water, the pH of the system is adjusted with 0.5N hydrochloric acid (preferably, adjusted to a pH 9-10), the resulting mixture is filtered, washed, and enriched, and the enriched solution is concentrated under reduced pressure, washed, and dried to obtain purified sinoporphyrin sodium.

According to the method for preparing sinoporphyrin sodium of the present disclosure, a mixture containing sinoporphyrin sodium as the target product is separated and enriched by using a specific ratio of methanol and water as eluents in a preparative high performance liquid chromatography process, thereby obtaining a high-purity product concentrate solution suitable for subsequent processing steps such as freeze drying.

In a preferred embodiment, in step (S4), the weight ratio of methanol to water for dissolving the compound of sinoporphyrin sodium in formula (I) is 1:(2-6), preferably, 1:(3-5).

In a preferred embodiment, in step (S4), methanol and water are used as eluents in the step of purification by preparative high performance liquid chromatography (HPLC).

In a preferred embodiment, step (S4) may further comprise a filtration step, wherein the filtration is performed at least once using a filter under positive pressure, preferably, 1-3 times, and more preferably, 2 times. Preferably, during the filtration process, washing is performed once with methanol and water, wherein the weight ratio of methanol to water is 1:(2.5-4.5), preferably, 1:(3-4). In a preferred embodiment, the filter is a pipeline filter, which may be a straight-through type, T-type, Y-type, etc., preferably, a straight-through type. The pore size of the pipeline filter is 0.2-0.6 μm, preferably, 0.4-0.5 μm.

In a preferred embodiment, in step (S4), the solution enriched by preparative high performance liquid chromatography (HPLC) is washed with acetonitrile and water.

In a preferred embodiment, the mixture obtained by dissolving the compound of sinoporphyrin sodium in formula (I) in step (S3) is enriched by preparative high performance liquid chromatography (HPLC) at least once, preferably, 1-3 times, more preferably, 2 times, wherein the eluents are methanol and water. Specifically, the volume ratio of methanol (A) and water (B) by the elution gradient used for enrichment by preparative high performance liquid chromatography (HPLC) is A/B=10:90 and 70:30, respectively, and the flow rate A+B is 200-300 mL/min, preferably, 220-280 mL/min.

In a preferred embodiment, before enriching the mixture obtained by dissolving the compound in formula (I) in step (S3), the mixture may be subjected to preparative high performance liquid chromatography (HPLC) at least once, preferably, 1-3 times, more preferably, 2 times, wherein the eluents are methanol and water. Specifically, the volume ratio of methanol (A) to water (B) by the elution gradient used in the chromatographic separation prior to enrichment by preparative high performance liquid chromatography (HPLC) is A/B=45:55, 100:0 and 10:90 successively, wherein the flow rate A+B is 100-180 mL/min, preferably, 110-150 mL/min when A/B=45:55, and A+B is 200-300 mL/min, preferably, 220-280 mL/min, when A/B=100:0 and 10:90.

In step (S4), separation and enrichment by preparative high performance liquid chromatography (HPLC) is performed using a wet packing method with a methanol (A)/water (B) ratio of A/B=10:90 and column equilibration for 10 min.

According to the method for preparing sinoporphyrin sodium of this application, step (S4) may further comprise a vacuum concentration step after purification by preparative high performance liquid chromatography. There are no particular limitations on the apparatus used for vacuum concentration, as long as it can achieve the purpose of vacuum concentration. Preferably, the vacuum concentration is carried out using a rotary evaporator at ≤15° C., and the internal pressure of the rotary evaporator is −0.09 MPa to 0 MPa.

In a preferred embodiment, step (S4) may further comprise a drying step, wherein the drying is freeze-drying and is carried out using equipment conventional for freeze-drying in the art, preferably, a plate freeze dryer. Preferably, the moisture content (% w/w) of the sinoporphyrin sodium obtained after the drying step is ≤10%.

In this application, the determination of moisture content is a method well known to those skilled in the art, such as the Karl Fischer method for determining water content.

A third aspect of the present disclosure provides the compound of protoporphyrin IX dimethyl ester in formula (III), the compound of bis[1-[6,7-dipropionate methyl-1,3,5,8-tetramethyl-2-vinyl-4-porphyrin]ethyl] ether in formula (II), and the compound of sinoporphyrin sodium in formula (I), prepared according to the method of the second aspect of the present disclosure.

A fourth aspect of the disclosure provides a pharmaceutical composition for photodynamic therapy, which comprises sinoporphyrin sodium prepared according to the method of the second aspect of the invention and one or more pharmaceutically acceptable excipients.

The compound of sinoporphyrin sodium in formula (I) of the present disclosure can be formulated into any suitable galenical preparation and can be administered by any suitable manner. For example, the the compound of sinoporphyrin sodium in formula (I) of the present disclosure can be formulated into solutions, suspensions, emulsions, lyophilized formulations, etc., for injection (e.g., intra-arterial, intravenous, intramuscular, subcutaneous, intraperitoneal injection, etc.) or infusion; formulated into tablets, solutions, capsules, etc., for oral administration; formulated into ointments, creams, suppositories, patches, etc., for topical application; and formulated into aerosols, sprays, powders, etc., for inhalation application.

The method for formulating the compound of sinoporphyrin sodium in formula (I) of the present disclosure into a galenical preparation, as well as the pharmaceutically acceptable excipients that may be used, are conventional methods and known excipients well known to those skilled in the art. For example, descriptions for such galenical preparation and suitable excipients are found in the following literature: Luo Mingsheng and Gao Tianhui, eds., “Complete Compendium of Pharmaceutical Excipients”, 2nd edition, Sichuan Science and Technology Press. Those skilled in the art can modify the formulations within the scope of the teachings of this specification to provide various formulations for specific manners of administration without destabilizing the compounds of the present disclosure or impairing their therapeutic activity.

A fifth aspect of the disclosure provides the use of sinoporphyrin sodium prepared according to the method of the second aspect of the disclosure in the preparation of a medicament used as a photosensitizer in photodynamic therapy.

It should be noted that all characteristics, parameters, conditions, other elements and combinations thereof described in this specification regarding the preparation methods and products of sinoporphyrin sodium and intermediates used in its synthesis are applicable to pharmaceutical compositions and uses containing it.

The present disclosure will be further described in more detail with reference to the following examples. However, these examples are merely exemplary and should not be construed as limiting the scope of the disclosure in any way.

### EXAMPLE

Unless otherwise specified, all reagents or instruments used are commercially available, conventional products commonly used in this field.

### Example 1

### S1. Preparation of the Compound of Protoporphyrin IX Dimethyl Ester in Formula (III)

Under a nitrogen atmosphere, 362 volumns (590 g at standard conditions of 0° C., 101.325 kPa) of hydrogen chloride gas (purchased from Zibo Wandali Special Gas Company, purity: ≥99.8%) was introduced into a reaction vessel (Jiangsu Purui Technology, 20 L of glass reaction vessel; 0.01 MPa of internal pressure) containing 4.95 L of methanol at 15° C., and stirred for 10-30 minutes at this temperature. Then, at 25° C., 4.98 L of dichloromethane, 500 g of hemin chloride (Wuhan Dahua Weiye Pharmaceutical Chemical Co., Ltd.), and 25.5 g of reduced iron powder were added, with the reduced iron powder added in three batches of 8.5 g each, and stirred for 3 hours.

After the temperature of the reaction vessel is cooled to 15° C., 1000 g of triethylamine was added dropwisely to adjust the pH of the system to pH=2. At 25° C., the resulting mixture was divided into two batches, with 2.5 L of water (self-prepared purified water) added to each batch and stirred for 10 minutes. After settling and separating into layers, the organic and aqueous phases were transferred and combined seperately. Then, 300 g of triethylamine was added dropwisely to the combined organic phase to adjust the pH of the system to pH=8. The resulting mixture after pH adjustment was divided into two batches, with 2.5 L of water added to each batch and stirred for 10 minutes. After settling and separating into layers, the organic and aqueous phases were transferred and combined seperately.

The combined organic phase obtained above was filtered through a positive pressure stainless steel filter plate filter (10 μm of pore size). The resulting filtrate was concentrated to ⅓ of its original volume under reduced pressure at 20° C. in a reaction vessel equipped with a stirrer, a temperature control device, a pressure reducing device, and a secondary condenser. Then, 0.94 L of dichloromethane and 4.42 L of methanol were added for washing. The resulting mixed solution was centrifuged using a centrifuge (Jiangsu Saideli Pharmaceutical Machinery Manufacturing Co., Ltd., LLGZ800 model), and the resulting solid was dried at 25° C. in a vacuum drying oven (Shanghai Haixiang Instrument Equipment Factory, DZF-6090) to obtain 440 g of purplish-brown crystalline powder protoporphyrin IX dimethyl ester (yield 97% (by weight, the same below), purity 97%, melting point 226° C.). The purity of protoporphyrin IX dimethyl ester was determined by HPLC (column: Waters Sunfire C18; column temperature: 30° C.; mobile phase A: 0.05% trifluoroacetic acid aqueous solution, mobile phase B: 0.05% trifluoroacetic acid acetonitrile solution, A/B=5/95 and 95/5, flow rate A+B=1.0 mL/min).

### S2. Preparation of the Compound of Ether-Bonded Porphyrin Dimer Ester in Formula (II)

Under a nitrogen atmosphere, 130.7 volumes (472 g at standard conditions of 0° C., 101.325 kPa) of hydrogen bromide gas (purchased from Chengdu Taiyu Special Gas Company, purity: ≥99%) and 6.8 volumes (9.8 g at standard conditions of 0° C., 101.325 kPa) of oxygen (molar content in hydrogen bromide gas is approximately 5%) were introduced into a reaction vessel (Jiangsu Nantong Purui Technology, 30 L glass reaction vessel) containing 9.54 L of dichloromethane, and stirred for 10-30 minutes. 440 g of the protoporphyrin IX dimethyl ester prepared in Example S1 was added, and the reaction was carried out at 0° C. for 16 hours. 4.4 L of water was added at 25° C., and the mixture was stirred for 2 hours.

4400 g of 7% sodium bicarbonate solution was added dropwisely to the reaction vessel at 25° C. in order to adjust the pH of the system to pH=7. The resulting mixture was divided into two batches, with 1.98 L of dichloromethane and 2.2 L of water added to each batch. After stirring and settling, the organic and aqueous phases were transferred and combined separately. Then, 4.3 L of dichloromethane was added to the combined aqueous phase, and after stirring and settling, the organic and aqueous phases were transferred separately. The transferred organic phase was then combined with the organic phase obtained after the first extraction. The combined organic phase after the second extraction was filtered through a 10 μm stainless steel filter. The resulting filtrate was then concentrated under reduced pressure at 20° C. to a final volume of 2 L in a reaction vessel equipped with a stirrer, a temperature control device, a reduced pressure device (0.01 MPa), and a secondary condenser. 662 mL of dichloromethane was added to obtain an ether-bonded porphyrin dimer ester-dichloromethane solution.

The above-mentioned ether-bonded porphyrin dimer ester-dichloromethane solution was purified once by preparative medium-pressure liquid chromatography (manufacturer: Jiangsu Hanbang; model: DAC450; packing material: FUJI (15 μm); mobile phase A: 10% acetone solution; mobile phase B: dichloromethane) using the cleaning balance column parameters and separation parameters shown in Table 1 (elution gradient using (1) and (2) in series). The resulting product solution containing ether-bonded porphyrin dimer ester was then purified a second time using the cleaning balance column parameters and separation parameters shown in Table 1 (elution gradient using (3)), to yield a product solution containing ether-bonded porphyrin dimer ester, a crossed solution containing ether-bonded porphyrin dimer ester, and a product-free solution containing porphyrin derivative impurities. The product solution containing ether-bonded porphyrin dimer ester obtained after the second purification was then concentrated under reduced pressure at 20° C. in a reaction vessel equipped with a stirrer, a temperature control device, a pressure reducing device, and a secondary condenser. After washing with dichloromethane, it was concentrated again under reduced pressure to 2 L. The resulting ether-bonded porphyrin dimer ester-dichloromethane concentrate solution was dried at 30° C. in a vacuum drying oven (Shanghai Haixiang Instrument Equipment Factory, DZF-6090) to obtain 239 g of ether-bonded porphyrin dimer ester in the form of a dark red powder, with a yield of 55.2% and a purity of 98%. The purity of the ether-bonded porphyrin dimer ester was analyzed by HPLC (column: Waters Xbridge C₈; column temperature: 30° C.; mobile phase A: 0.05% trifluoroacetic acid aqueous solution, mobile phase B: 0.05% trifluoroacetic acid acetonitrile solution, A/B=70/30, 25/75 and 5/95, flow rate A+B=0.8 mL/min).

TABLE 1
1. Cleaning column parameters

| Mobile phase A | Solvent: 10% acetone solution |
|---|---|
| Mobile phase B | Solvent: dichloromethane |
| Ratio A/B | 50/50 |
| Flow rate (L/min) | 6.5 L/min |
| Time/min | 20 |

2. Balance column parameters

| | |
|---|---|
| Mobile phase A | Solvent: dichloromethane |
| Flow rate (L/min) | 6.5 L/min |
| Time/min | 40 |

3. Separation parameters

| Device | DAC450 | | | |
|---|---|---|---|---|
| Packing material | 15 μm silicone | | | |
| Mobile phase A | Solvent: 10% acetone solution | | | |
| Mobile phase B | Solvent: dichloromethane | | | |
| Gradient elution | Time/min | A % | B % | Flow rate A + B |
| | | | | (L/min) |
| (1) | 0 | 10 | 90 | 6.5 |
| | 100 | 10 | 90 | 6.5 |
| (2) | 0 | 20 | 80 | 6.5 |
| | 100 | 20 | 80 | 6.5 |
| (3) | 0 | 16 | 84 | 4.5 |
| | 60 | 16 | 84 | 4.5 |
| | 60.1 | 0 | 100 | 6.5 |
| | 80 | 0 | 100 | 6.5 |

| | |
|---|---|
| Detection | 254 nm, 380 nm |
| wavelength | |

### S3. Preparation of the Compound of Sinoporphyrin Sodium in Formula (I)

S3-a: Under a nitrogen atmosphere, 239 g of the ether-bonded porphyrin dimer ester prepared in Example S2 was dissolved in 4.78 L of dichloromethane and mixed with a sodium hydroxide methanol solution pre-prepared with 3.63 L of methanol and 167.3 g of sodium hydroxide in a reaction vessel (Nantong Purui Technology, 10 L glass reaction vessel), then stirred at 20° C. for 16 hours and then allowed to settle. The solution was then vacuum filtered through a tower filter (Tianjin Jinteng Experimental Equipment Co., Ltd., 10 L glass filter device, 10 μm membrane pore size), washed with 303 mL of ethanol, and after concentration, the resulting ethanol solution containing sinoporphyrin sodium was dried at 35° C. in a vacuum drying oven (Shanghai Haixiang Instrument Equipment Factory, DZF-6090) to obtain 228 g of sinoporphyrin sodium with a purity of 96% and a yield of 95%. The purity of sinoporphyrin sodium was analyzed by HPLC (column: Waters Xbridge C₈; column temperature: 30° C.; mobile phase A: 0.05% trifluoroacetic acid aqueous solution, mobile phase B: 0.05% trifluoroacetic acid acetonitrile solution, A/B=70/30, 25/75 and 5/95, flow rate A+B=0.8 mL/min).

S3-b: Under a nitrogen atmosphere, 2 L of the to-be-dried ether-bonded porphyrin dimer ester-dichloromethane concentrate solution prepared in Example S2 was mixed with a sodium hydroxide methanol solution pre-prepared with 3.63 L of methanol and 167.3 g of sodium hydroxide in a reaction vessel (Nantong Purui Technology, 30 L glass reaction vessel), then stirred at 20° C. for 16 hours and then allowed to settle. The solution was then vacumm filtered through a tower filter (Tianjin Jinteng Experimental Equipment Co., Ltd., 10 L glass filter device, 10 μm membrane pore size), washed with 320 mL of ethanol, and the resulting ethanol solution containing sinoporphyrin sodium was dried at 35° C. in a vacuum drying oven (Shanghai Haixiang Instrument Equipment Factory, DZF-6090) to obtain 220 g of sinoporphyrin sodium with a purity of 95% and a yield of 91.5%. The residual dichloromethane (% w/w) was 0.27%, and the residual methanol (% w/w) was 0.15%.

### S4. Purification Step

5.68 L of methanol and 18 L of water were mixed in a reaction vessel. Under a nitrogen atmosphere, 228 g of the compound of sinoporphyrin sodium in formula (I) prepared in Example S3-a was added with stirring at 25° C. to dissolve it completely. 36 g of 0.5N hydrochloric acid was added dropwisely and in batches to adjust the pH of the system to 9. The resulting mixture was filtered through a pipeline filter (Millibor, pore size: 0.45 μm). The filtrate was subjected to two chromatographic separations using preparative high performance liquid chromatography (manufacturer: Jiangsu Hanbang; model: DAC80; column diameter: 8 cm; mobile phase A: methanol 45%; mobile phase B: purified water 55%) with the parameters shown in Table 2 (using elution gradient (1)), to yield a product solution containing sinoporphyrin sodium with a purity of 98%, a crossed solution containing sinoporphyrin sodium, and a product-free solution containing other impurities.

The product solution containing sinoporphyrin sodium was subjected to preparative high performance liquid chromatography (HPLC) (manufacturer: Jiangsu Hanbang; model: DAC80; column diameter: 8 cm; mobile phase A: methanol; mobile phase B: purified water) using the parameters shown in Table 2 for enrichment, resulting in an enriched product solution containing sinoporphyrin sodium. The product solution was then concentrated under reduced pressure at 10° C. using a rotary evaporator (internal pressure: −0.06 MPa) (four times, the first wash with 4 L of water and 1.7 L of acetonitrile, followed by each subsequent wash with acetonitrile) to a final volume of 2 L, yielding an acetonitrile aqueous concentrate containing sinoporphyrin sodium. The acetonitrile aqueous concentrate containing sinoporphyrin sodium was then subjected to preparative HPLC with the same parameters as the first enrichment for a secondary enrichment, resulting in a product solution containing sinoporphyrin sodium. The product solution after the second enrichment was concentrated in batches under reduced pressure at 10° C. by a rotary evaporator (−0.06 MPa of internal pressure). It was then washed with 2.7 L of water and 2.7 L of acetonitrile and concentrated under reduced pressure to 2 L, and then combined to obtain the concentrated acetonitrile aqueous solution containing sinoporphyrin sodium.

TABLE 2
1. Parameters used for separation

| Device | Preparative liquid phase system DAC80 | | | |
|---|---|---|---|---|
| Packing material | Nano Micro C18 | | | |
| Mobile phase A | methanol | | | |
| Mobile phase B | water | | | |
| Injection volume | 100-500 mL | | | |
| | Time/min | A % | B % | Flow rate A + B |
| | | | | (mL/min) |
| Gradient elution (1) | 0 | 45 | 55 | 130 |
| | 66 | 45 | 55 | 130 |
| | 66.1 | 100 | 0 | 250 |
| | 75 | 100 | 0 | 250 |
| | 75.1 | 10 | 90 | 250 |
| | 80 | 10 | 90 | 250 |

| | |
|---|---|
| Detection | 254 nm, 380 nm |
| wavelength | |

2. Parameters for enrichment

| Device | Preparative liquid phase system DAC80 | | | |
|---|---|---|---|---|
| Mobile phase A | Methanol | | | |
| Mobile phase B | Water | | | |
| Injection volume | First | 10000-15000 mL | | |
| | Second | 1000-2000 mL | | |
| | Time/min | A % | B % | Flow rate A + B |
| | | | | (mL/min) |
| Gradient elution (2) | 0 | 10 | 90 | 250 |
| | 5 | 10 | 90 | 250 |
| | 5.1 | 70 | 30 | 250 |
| | 25 | 70 | 30 | 250 |
| | 25.1 | 10 | 90 | 250 |
| | 30 | 10 | 90 | 250 |

| | |
|---|---|
| Detection | 254 nm, 380 nm |
| wavelength | |

The acetonitrile aqueous concentrate solution containing sinoporphyrin sodium obtained after the second enrichment was freeze-dried using a plate freeze dryer (Shanghai Dongfulong Technology Co., Ltd., 2 m²) to obtain 194 g of purified sinoporphyrin sodium with a purity of 99.6%, a purification yield of 85%, and a moisture content of 3.3%.

Calculations show that the total weight yield of the compound of sinoporphyrin sodium in formula (I) according to the present disclosure is 51%. The total weight yield of sinoporphyrin sodium synthesized from the compound of protoporphyrin IX dimethyl ester in formula (III) is 52.4%.

¹H NMR (δ, ppm): 10.30, 10.08, 9.99, 9.93 (each s, 1H, meso H); 8.28 (m, 1H, CH═CH₂), 6.41, 6.18 (each d, 1H, CH═CH₂); 6.25 (q, 1H, CH(OH)CH₃); 4.33 (m, 2H, 2CH₂CH₂CO₂CH₃); 3.69 (s, 9H, 2CO₂CH₃ and 1CH₃); 3.55, 3.51, 3.40 (each s, 3H, 3CH₃); 3.21 (t, 4H, 2CH₂CH₂CO₂CH₃); 2.03 (d, 3H, CH(OH)CH₃).

AccuTOF CS cold-spray mass spectrometer (JEOL, Japan); CSI ion source; spray temperature: room temperature; desolventization temperature: 250° C.; solvent: methanol; concentration: 40 ng/μL.

The cold-spray MS data of sinoporphyrin sodium obtained through Example 1 is shown below.

Cold-spray MS m/z: 1253.44 [M+Na]⁺, 1231.96 [M+H]⁺, 1209.47 [M+2H−Na]⁺, 1187.48 [M+3H−2Na]⁺, 1165.50 [M+4H−3Na]⁺, 1143.52 [M+5H−4Na]⁺.

High-resolution electrospray ionization mass spectrometry (HR-ESI-MS) yielded a quasi-molecular ion peak [M+Na]⁺ at m/z 1253.44231, corresponding to the molecular formula C₆₈H₆₆N₈O₉Na₄+Na (calculated value 1253.44412).

Both the synthesis reaction and MS analysis results indicate that the final product is sinoporphyrin sodium, namely, bis[1-[6,7-dipropionate-1,3,5,8-tetramethyl-2-vinyl-4-porphyrin]ethyl] ether.

### Example 2

### Preparation of Lyophilized Solution of Sinoporphyrin Sodium for Injection

10 g of the sinoporphyrin sodium raw material prepared in Example 1 was weighed and put in a light-proof glass container, and dissolved it in water for injection to achieve a concentration of 2.5 mg/mL. The solution was filtered under pressure through a stainless steel bacterial filter, firstly through a prefiltering membrane with a pore size of 0.45 mm, then a sterile membrane with a pore size of 0.2 μm. The solution was quantitatively dispensed into 10 mL glass ampoules in a sterile operating room, with a dispensing volume of 4 mL, and freeze-dried using a freeze drier under vacuum at −20° C. to obtain the lyophilized solution for injection.

### Comparative Example 1

### 1. Preparation of ether-bonded porphyrin dimer ester (i.e., bis[1-[6,7-dipropionate methyl-1,3,5,8-tetramethyl-2-vinyl-4-porphyrin]ethyl] ether)

### (a) Preparation of Protoporphyrin Derivative Dimethyl Ester

100 g of protoporphyrin IX dimethyl ester was added to 500 mL of hydrochloric acid to complete dissolution with stirring, and stirred with a water bath at 25° C. and reacted for 6 hours, added 2800 mL of 20% sodium hydroxide solution, with pH=13. The reaction was allowed to settle for 1 hour, then 500 mL of acetic acid was added, further reacted at pH=5 for 30 minutes, vacumm filtered and washed the residue with water, drained and dried in a vacuum drying oven to obtain 95 g brown solid. The above 95 g solid was dissolved in 1000 mL of 5% sulfuric acid methanol solution, stirred and reacted for 30 minutes at room temperature. Then, (NH₄)₂CO₃ was added to neutralize to pH 7, and the solution was concentrated under reduced pressure. The concentrate solution was extracted with dichloromethane, washed with water, dehydrated with anhydrous sodium sulfate, filtered, and the dichloromethane was recovered under reduced pressure to obtain the protoporphyrin derivative dimethyl ester. The protoporphyrin derivative dimethyl ester was dissolved in an appropriate amount of acetone, and subjected to chromatography through a silica gel column (1600 g silica gel, 200-400 mesh, pre-balanced with 0.2% methanol dichloromethane solution) by using 0.2% methanol dichloromethane solution, to obtain 78 g of protoporphyrin derivative dimethyl ester 4(2)-(1-hydroxyethyl)-6,7-bis[2-(methoxycarbonyl)ethyl]-1,3,5,8-tetramethyl-2(4)-vinylporphyrin, yielding 75.7%.

### (b) Separation and Purification of Protoporphyrin Derivative Dimethyl Ester

78 g of the protoporphyrin derivative dimethyl ester 4(2)-(1-hydroxyethyl)-6,7-bis[2-(methoxycarbonyl)ethyl]-1,3,5,8-tetramethyl-2(4)-vinylporphyrin prepared above was dissolved in 400 mL of dichloromethane, and subjected to chromatography through a glass chromatography column (1000 g silica gel, fineness 160-200 mesh, dichloromethane as eluent, column diameter 60 mm), to obtain 50 g of 4-(1-hydroxyethyl)-6,7-bis[2-(methoxycarbonyl)ethyl]-1,3,5,8-tetramethyl-2-vinylporphyrin with a purity of 98% and a yield of 64.1%.

### (c) Preparation of Ether-Bonded Porphyrin Dimer Methyl Ester

50 g of 4-(1-hydroxyethyl)-6,7-bis[2-(methoxycarbonyl)ethyl]-1,3,5,8-tetramethyl-2-vinylporphyrin was dissolved in 3 L of anhydrous dichloromethane, 1 L of dichloromethane solution saturated with hydrogen bromide gas was added, and reacted in the dark under sealed conditions after the mixture was shaken well. The reaction was terminated by adding water, and the dichloromethane solution was separated, washed with water, dehydrated with anhydrous sodium sulfate, the dichloromethane was recovered under reduced pressure, and passed through silica gel column (eluting with acetone and dichloromethane (1:60)) to separate and purify, yielding 30 g of ether-bonded porphyrin dimer methyl ester, with a purity of 94% and a yield of 62.1%.

### 2. Preparation of Sinoporphyrin Sodium

30 g of ether-bonded porphyrin dimer ester was dissolved in 3.6 L of tetrahydrofuran, 2.4 L of 0.1 mol/L sodium hydroxide aqueous solution was added, and heated under reflux in an oil bath at 80° C. in the dark for 8 hours, the tetrahydrofuran aqueous solution was evaporated under reduced pressure. The dried reaction product was loaded into a reflux eluent, and an appropriate amount of anhydrous ethanol was added for reflux and elution to remove the remaining sodium hydroxide and other impurities, yielding 18 g of purified sinoporphyrin sodium, with a purity of 98% and a yield of 62.2%.

The total weight yield of the method in Comparative Example 1 was 18.8%. Surprisingly, compared with the sinoporphyrin sodium prepared by Comparative Example 1 using existing methods, the sinoporphyrin sodium prepared by the method of the present disclosure exhibits a significantly improved yield. For example, the total weight yield of sinoporphyrin sodium prepared from protoporphyrin IX dimethyl ester as a starting material is 52.4% (significantly higher than 18.8% in Comparative Example 1), while the purity of the obtained sinoporphyrin sodium reaches 99.6%. Such high purity is beneficial for its application in demanding fields such as antitumor, antibacterial, and the development of therapeutic and diagnostic reagents. Furthermore, the method for preparing sinoporphyrin sodium according to the present disclosure uses inexpensive and readily available raw materials, has low equipment demands, and employs mild reaction conditions. It can obtain various intermediates with high quality (e.g., good yield and purity) suitable for the synthesis of sinoporphyrin sodium, thus making it particularly suitable for the industrial production of sinoporphyrin sodium.

What is claimed is:

1. A method for preparing a compound of bis[1-[6,7-dipropionate methyl-1,3,5,8-tetramethyl-2-vinyl-4-porphyrin]ethyl] ether in formula (II), the method comprising: 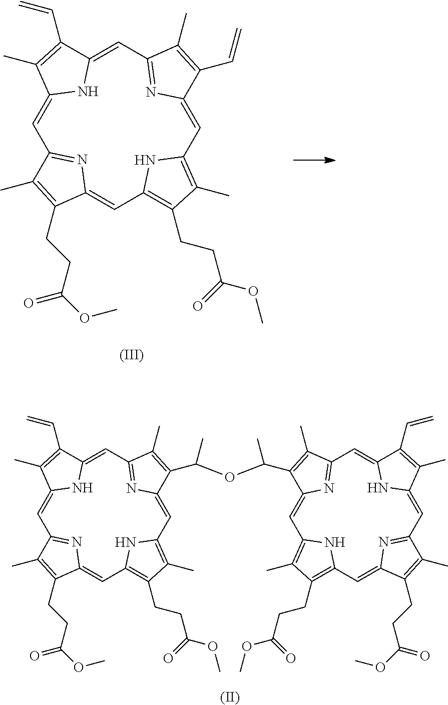
a compound of protoporphyrin IX dimethyl ester in formula (III) is reacted at −10-10° C. in the presence of haloalkane solvent, hydrogen bromide gas, and oxygen, to obtain the compound of bis[1-[6,7-dipropionate methyl-1,3,5,8-tetramethyl-2-vinyl-4-porphyrin]ethyl] ether in formula (II).

2. The method according to claim 1, wherein the haloalkane solvent is selected from C₁-C₃ haloalkane, preferably, selected from at least one of dichloromethane, dichloroethane, trichloromethane, carbon tetrachloride, and dibromomethane, and more preferably, dichloromethane.

3. The method according to claim 1, wherein a weight ratio of the compound of protoporphyrin IX dimethyl ester in formula (III) to hydrogen bromide gas is 1:(0.7-1.4), preferably, 1:(0.75-1.3), and more preferably, 1:(0.8-1.2); preferably, a molar content of oxygen in the hydrogen bromide gas in a reaction system is 4%-6%, preferably, 4.5%-5.5%.

4. A method for preparing a compound of sinoporphyrin sodium in formula (I), the method comprising the following steps: 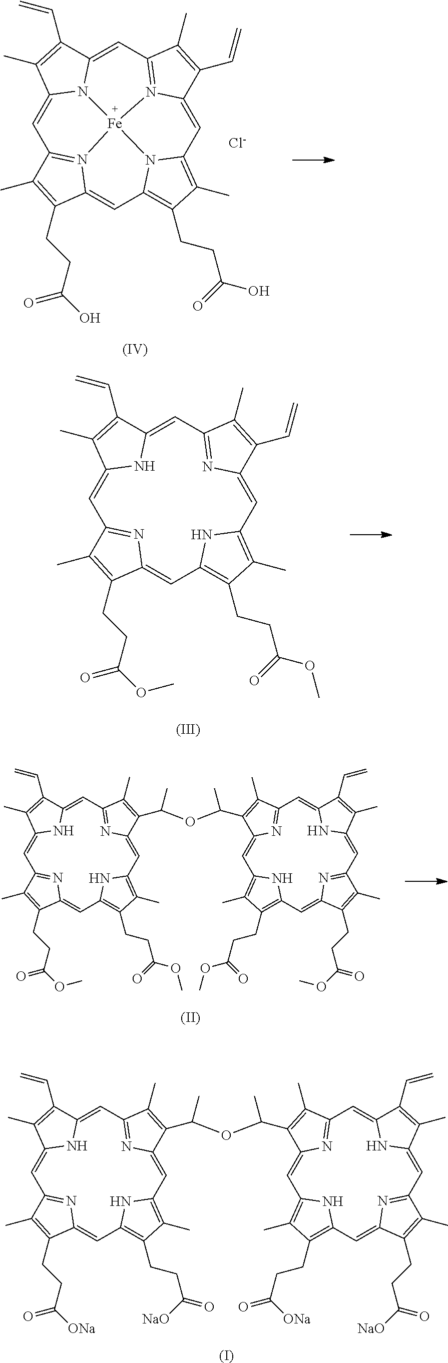
(S1) a compound of hemin chloride in formula (IV) is reacted with methanol and reduced iron powder in the presence of haloalkane solvent and hydrogen chloride gas, to obtain a compound of protoporphyrin IX dimethyl ester in formula (III);
(S2) the compound of bis[1-[6,7-dipropionate methyl-1,3,5,8-tetramethyl-2-vinyl-4-porphyrin]ethyl] ether in formula (II) is obtained from the compound in formula (III) by the method of claim 1;
(S3) the compound of bis[1-[6,7-dipropionate methyl-1,3,5,8-tetramethyl-2-vinyl-4-porphyrin]ethyl] ether in formula (II) is mixed and reacted with sodium hydroxide C₁-C₃ fatty alcohol solution in the presence of haloalkane solvent, to obtain the compound of sinoporphyrin sodium in formula (I).

5. The method according to claim 4, wherein the haloalkane solvent is selected from C₁-C₃ haloalkane, preferably, selected from at least one of dichloromethane, dichloroethane, trichloromethane, carbon tetrachloride, and dibromomethane, and more preferably dichloromethane; and the C₁-C₃ fatty alcohol is selected from methanol, ethanol, propanol, or mixtures thereof, preferably, methanol.

6. The method according to claim 4, wherein the method further comprises a step (S4) of purifying by preparative high performance liquid chromatography.

7. The method according to claim 4, wherein, in step (S1), the reaction is carried out at 18-30° C., preferably, at 20-30° C. for 1-7 hours, preferably, 2-6.5 hours, and more preferably, 3-6 hours; preferably, a weight ratio of the compound of hemin chloride in formula (IV), reduced iron powder, hydrogen chloride gas and methanol is 1:(0.045-0.065):(1.08-1.35):(7.7-8.0), preferably, 1:(0.048-0.06):(1.1-1.32):(7.8-8.0), and more preferably, 1:(0.05-0.06):(1.1-1.3):(7.8-7.9); preferably, the reduced iron powder is added in batches.

8. The method according to claim 4, wherein, in step (S3), the reaction is carried out at 15-30° C., preferably, 15-25° C., for 6-18 hours, preferably, 8-16 hours; preferably, a weight ratio of the compound in formula (II) to the sodium hydroxide C₁-C₃ fatty alcohol solution is 1:(10-15), preferably, 1:(11-14), wherein a weight ratio of sodium hydroxide to C₁-C₃ fatty alcohol is 1:(13-22), preferably, 1:(15-20).

9. The method according to claim 2, wherein a weight ratio of the compound of protoporphyrin IX dimethyl ester in formula (III) to hydrogen bromide gas is 1:(0.7-1.4), preferably, 1:(0.75-1.3), and more preferably, 1:(0.8-1.2); preferably, a molar content of oxygen in the hydrogen bromide gas in a reaction system is 4%-6%, preferably, 4.5%-5.5%.

10. The method according to claim 5, wherein the method further comprises a step (S4) of purifying by preparative high performance liquid chromatography.

11. The method according to claim 5, wherein, in step (S1), the reaction is carried out at 18-30° C., preferably, at 20-30° C. for 1-7 hours, preferably, 2-6.5 hours, and more preferably, 3-6 hours; preferably, a weight ratio of the compound of hemin chloride in formula (IV), reduced iron powder, hydrogen chloride gas and methanol is 1:(0.045-0.065):(1.08-1.35):(7.7-8.0), preferably, 1:(0.048-0.06):(1.1-1.32):(7.8-8.0), and more preferably, 1:(0.05-0.06):(1.1-1.3):(7.8-7.9); preferably, the reduced iron powder is added in batches.

12. The method according to claim 5, wherein, in step (S3), the reaction is carried out at 15-30° C., preferably, 15-25° C., for 6-18 hours, preferably, 8-16 hours; preferably, a weight ratio of the compound in formula (II) to the sodium hydroxide C₁-C₃ fatty alcohol solution is 1:(10-15), preferably, 1:(11-14), wherein a weight ratio of sodium hydroxide to C₁-C₃ fatty alcohol is 1:(13-22), preferably, 1:(15-20).
